(19) United States
(12) Patent Application Publication
(10) Pub. No.: US 2024/0423845 A1
(43) Pub. Date: Dec. 26, 2024

(54) Blister Prevention System

(71) Applicant: 2think, LLC, Windsor, CO (US)

(72) Inventor: Kevin E. Willey, Windsor, CO (US)

(73) Assignee: 2think, LLC, Windsor, CO (US)

(21) Appl. No.: 18/214,357

(22) Filed: Jun. 26, 2023

(51) Int. Cl. A61F 13/06

(52) U.S. Cl. CPC A61F 13/066; A61F 2013/00404

(57) ABSTRACT

A friction barrier adapted to position between a support surface and a skin surface, wherein said friction barrier comprises a generally flat sheet material generally conformable by placement between said support surface and said skin surface, or a pre-formed material placed between said support surface and said skin surface, wherein said friction barrier has first side adapted to face said skin surface, the first side having a static coefficient of friction which reduces one or more of the mechanical factors contributing to blister development when the skin rubs against a support surface, including friction, pressure, shear, or moisture

### I. FIELD OF THE INVENTION

A friction barrier adapted to position between a support surface and a skin surface, wherein said friction barrier comprises a generally flat sheet material generally conformable by placement between said support surface and said skin surface, or a pre-formed material placed between said support surface and said skin surface, wherein said friction barrier has first side adapted to face said skin surface, the first side having a static coefficient of friction which reduces one or more of the mechanical factors contributing to blister development when the skin rubs against a support surface, including friction, pressure, shear, or moisture

### II. BACKGROUND OF THE INVENTION

Blisters are small pockets of fluid that usually form in the upper layers of skin once damaged. Blisters can develop anywhere on the body but are most common on the hands and feet. Blisters are common in people who are active, as illustrative examples: hikers, sports players and military personnel.

Friction is recognized as one of the four mechanical factors contributing to blister development, along with pressure, shear, and moisture. The effect of each of these factors toward blister development varies, but it is generally accepted that minimizing or eliminating friction between the skin surface and the support surface can reduce shear and thereby reduce the risk of blister development. Friction, where skin surface rubs against the support surface can create blisters independent of pressure and shear.

Friction can be quantified by measuring the coefficient of friction between surfaces. The coefficient of friction (fr) is a number that is the ratio of the resistive force of friction (Fr) divided by the normal or perpendicular force (N) pushing the objects together. It is represented by the equation: fr=Fr/N. In the case of viscoelastic materials, such as skin, however, Fr is not always constant, particularly at low loads. It is known that moist skin has a higher coefficient of friction (fr) than dry skin and that this makes it more susceptible to friction blisters.

Relatively recently, silicone dressings (such as: Mepilex® Foam Dressing from Mölnlycke) and liquid barrier films (such as: 3M™ Cavilon™ No Sting Barrier Film) have been promoted to reduce the risk of blister development. This is based on the concept that such dressings can reduce the shear stress and friction reaching the skin and reduce the risk of blister development.

A disadvantage of foam dressings and liquid barrier films is that both need to be applied and removed or shed from the skin, and foam dressings present the risk of additional trauma to the skin. Additionally, clinical studies have also been published on this topic, but they have found no statistical difference between the intervention and control groups. Brindle CT. Wegelin JA. Prophylactic dressing application to reduce pressure ulcer formation in cardiac surgery patients. J Wound Ostomy Continence Nurs 2012; 39:133. Liquid barrier films have been used for friction reduction for many years, but data supporting this practice are anecdotal. Additionally, the use of foam dressings and liquid barrier films must be weighed against other options that may be less expensive and/or less labor intensive.

There would be a substantial advantage in a friction barrier device effective in the prevention of skin blisters, that would not have to be applied and removed from or shed from the skin and could be reused.

### III. SUMMARY OF THE INVENTION

A broad object of particular embodiments of the invention can be to provide a friction barrier adapted to position between a support surface and a skin surface, where the friction barrier can be obtained in the form of a generally flat sheet material generally conformable by placement between the support surface and the skin surface, or obtained as a pre-formed material placed between the support surface and the skin surface, wherein the friction barrier first side adapted to face the skin surface having a static coefficient of friction (fr) of less than 0.2, and in particular embodiments less than 0.15.

Another broad object of particular embodiments of the invention can be a method of adapting a generally flat sheet material or pre-formed material for placement between a support surface and a skin surface as a friction barrier, wherein the flat sheet material generally conformable by placement between said support surface and said skin surface, wherein the flat sheet material or the pre-formed material having a first side facing the skin surface, the first side having a static coefficient of friction (fr) of less than 0.2 and in particular embodiments less than 0.15.

Another broad object of particular embodiments of the invention can be obtaining a friction barrier adapted for placement between a support surface and a skin surface, the friction barrier comprising a generally flat sheet material generally conformable by placement between the support surface and said skin surface, or obtaining a pre-formed material adapted for placement between the support surface and the skin surface, wherein the friction barrier has first side adapted to face the skin surface having a static coefficient of friction (fr) of less than 0.2 and in particular embodiments less than 0.15. The method can further include placing the friction barrier between the support surface and the skin surface, wherein the first side of said friction barrier faces the skin surface, and the second side of said friction barrier faces the support surface.

Naturally, further objects of the invention are disclosed throughout other areas of the specification, drawings, photographs, and claims.

### IV. BRIEF DESCRIPTION OF THE DRAWINGS

FIG. 1 illustrates a particular embodiment of a friction barrier inserted inside the heel portion of a shoe depicted in broken line and generally conformed to engage the skin surface of a heel of a foot depicted in broken line.

FIG. 2 is a top perspective view of the particular embodiment of a friction barrier depicted in FIG. 1 adapted to position between the support surface of the shoe and a heel skin surface, wherein the friction barrier comprises a generally flat sheet material generally conformable by placement between a support surface and a skin surface.

FIG. 3 is a bottom perspective bottom view of the particular embodiment of the friction barrier depicted in FIG. 2.

FIG. 4 is a top plan view of the particular embodiment of the friction barrier depicted in FIG. 2.

FIG. 5 is a bottom plan view of the particular embodiment of the friction barrier depicted in FIG. 2.

FIG. 6 is a first side elevation view of the particular embodiment of the friction barrier depicted in FIG. 2.

FIG. 7 is a second side elevation view of the particular embodiment of the friction barrier depicted in FIG. 2.

FIG. 8 is a first end elevation view of the particular embodiment of the friction barrier depicted in FIG. 2.

FIG. 9 is a second end elevation view of the particular embodiment of the friction barrier depicted in FIG. 2.

FIG. 10 illustrates another particular embodiment of a friction barrier inserted inside the forefoot portion of a shoe depicted in broken line and generally conformed to engage the skin surface of a forefoot of a foot depicted in broken line.

FIG. 11 is a top perspective view of a particular embodiment of the friction barrier depicted in FIG. 10 adapted to position between a support surface of the shoe and the skin surface of the forefoot of the foot, wherein the friction barrier comprises a generally flat sheet material generally conformable by placement between the support surface of the forefoot of the shoe and the skin surface of the forefoot.

FIG. 12 is a bottom perspective bottom view of the particular embodiment of the friction barrier depicted in FIG. 11.

FIG. 13 is a top plan view of the particular embodiment of the friction barrier depicted in FIG. 11.

FIG. 14 is a bottom plan view of the particular embodiment of the friction barrier depicted in FIG. 11.

FIG. 15 is a first side elevation view of the particular embodiment of the friction barrier depicted in FIG. 11.

FIG. 16 is a second side elevation view of the particular embodiment of the friction barrier depicted in FIG. 11.

FIG. 17 is a first end elevation view of the particular embodiment of the friction barrier depicted in FIG. 11.

FIG. 18 is a second end elevation view of the particular embodiment of the friction barrier depicted in FIG. 11.

FIG. 19 illustrates another particular embodiment of a friction barrier inserted inside the combination of the forefoot, the midfoot and the heel portion of a shoe depicted in broken line and generally conformed to engage the skin surface of the combination of the forefoot, the midfoot and the heel portion of a foot depicted in broken line.

FIG. 20 is a top perspective view of the particular embodiment of a friction barrier depicted in FIG. 19 adapted to position between the support surface of the shoe and combination of the forefoot, midfoot and heel skin surface, wherein the friction barrier comprises a generally flat sheet material generally conformable by placement between a support surface and a skin surface.

FIG. 21 is a bottom perspective bottom view of the particular embodiment of the friction barrier depicted in FIG. 20.

FIG. 22 is a top plan view of the particular embodiment of the friction barrier depicted in FIG. 19.

FIG. 23 is a bottom plan view of the particular embodiment of the friction barrier depicted in FIG. 20.

FIG. 24 is a first side elevation view of the particular embodiment of the friction barrier depicted in FIG. 20.

FIG. 25 is a second side elevation view of the particular embodiment of the friction barrier depicted in FIG. 20.

FIG. 26 is a first end elevation view of the particular embodiment of the friction barrier depicted in FIG. 20.

FIG. 27 is a second end elevation view of the particular embodiment of the friction barrier depicted in FIG. 20.

FIG. 28 illustrates another particular embodiment of a friction barrier inserted inside the heel portion of a shoe depicted in broken line and generally conformed to engage the skin surface of a heel of a foot depicted in broken line, wherein the friction barrier includes a first layer and a second layer, which may include first layer integrated with a second layer, or may be first layer discrete from the second layer.

FIG. 29 is a top perspective view of the particular embodiment of the friction barrier depicted in FIG. 28 adapted to position between the support surface of the shoe and a heel skin surface, wherein the friction barrier comprises two layers of generally flat sheet material generally conformable by placement between a support surface and a skin surface.

FIG. 30 is a top perspective exploded view of the particular embodiment of the friction barrier depicted in FIG. 29.

FIG. 31 is a bottom perspective exploded view of the particular embodiment of the friction barrier depicted in FIG. 29.

FIG. 32 is first side elevation view of the particular embodiment of the friction barrier depicted in FIG. 29 with an enlargement of a portion of FIG. 32 illustrating raised pattern element to reduce friction forces between a first layer and a second layer of the friction barrier to enhance movement of the first layer relative to the second layer of the friction barrier.

FIG. 33 is bar graph illustrating the relative coefficient of friction of a variety of materials which can be useful in making embodiments of the inventive friction barrier.

### V. DETAILED DESCRIPTION OF THE INVENTION

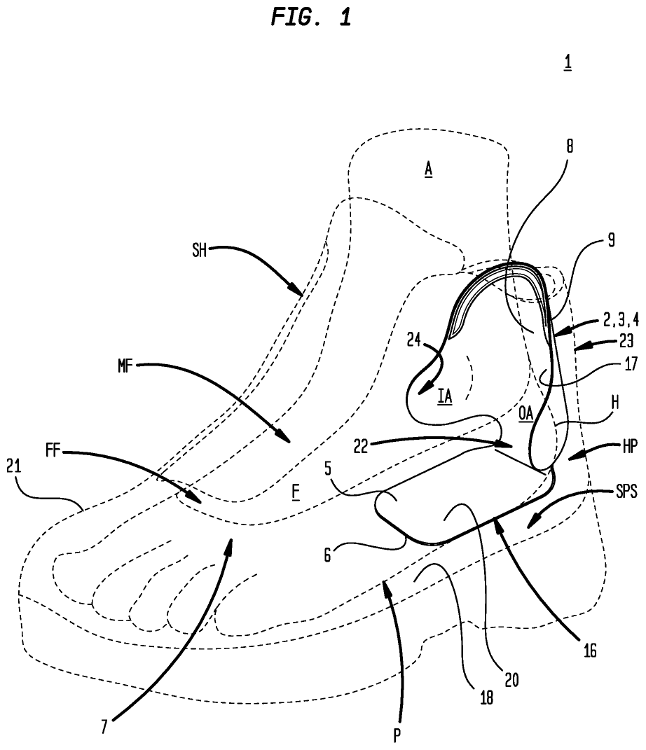

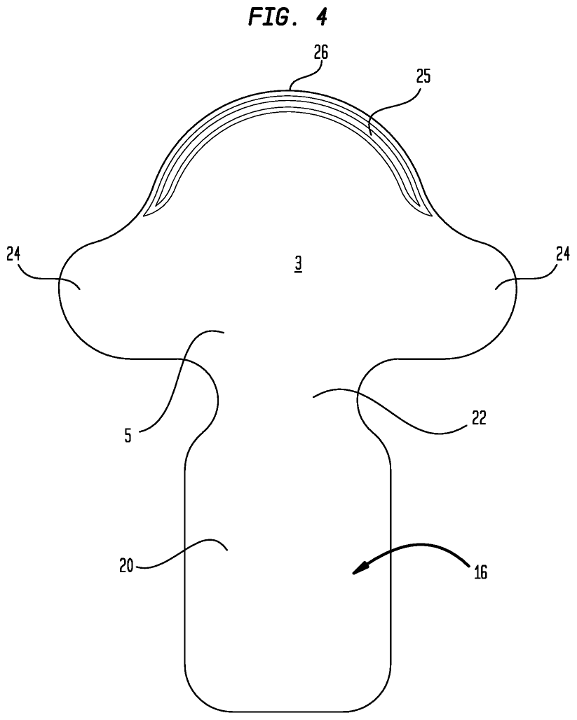

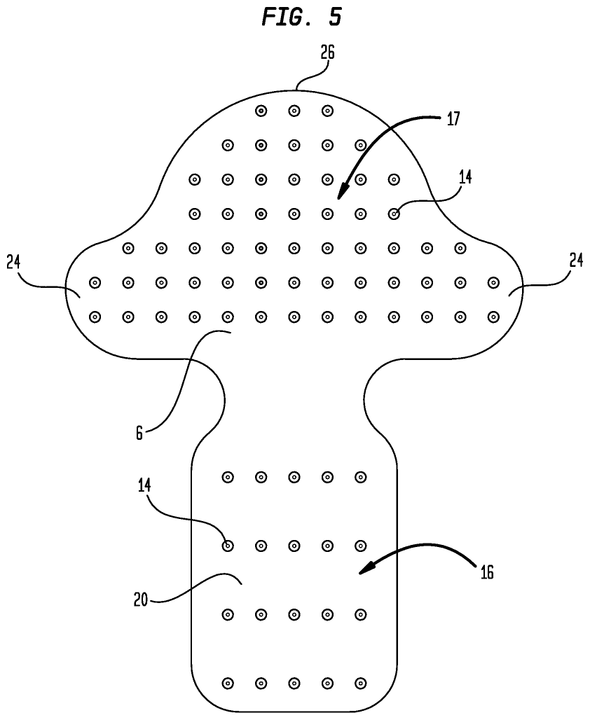

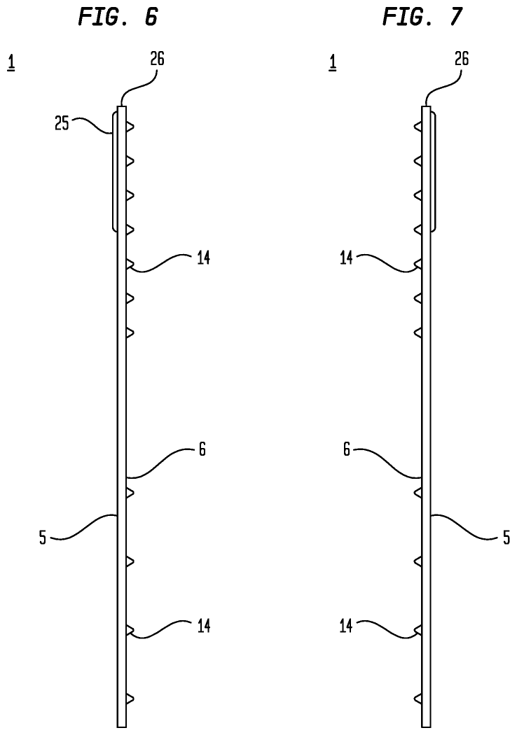

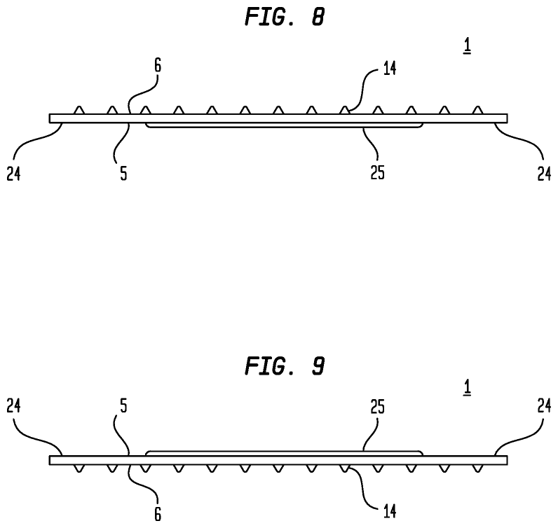

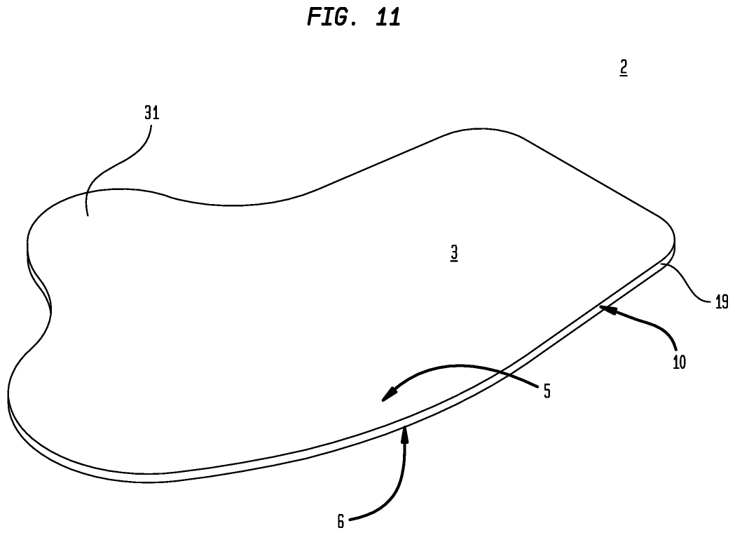

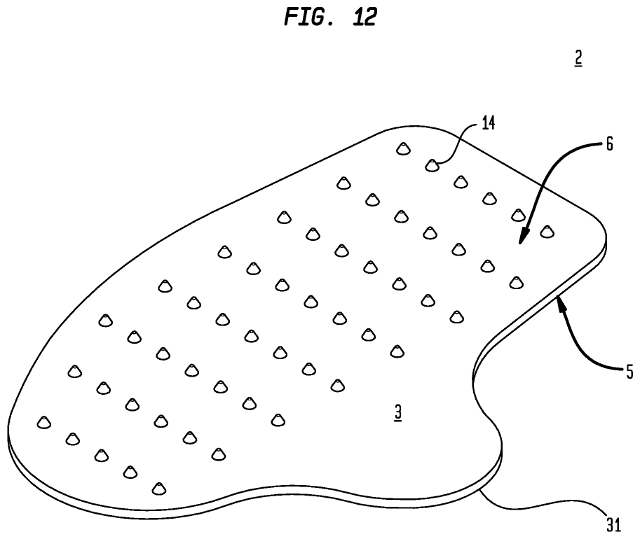

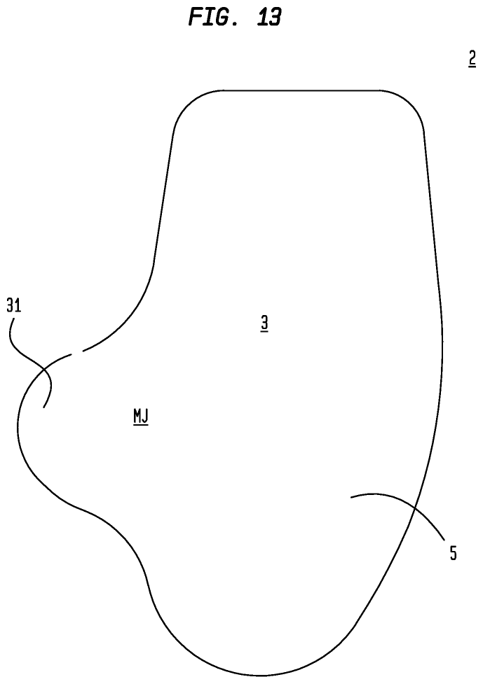

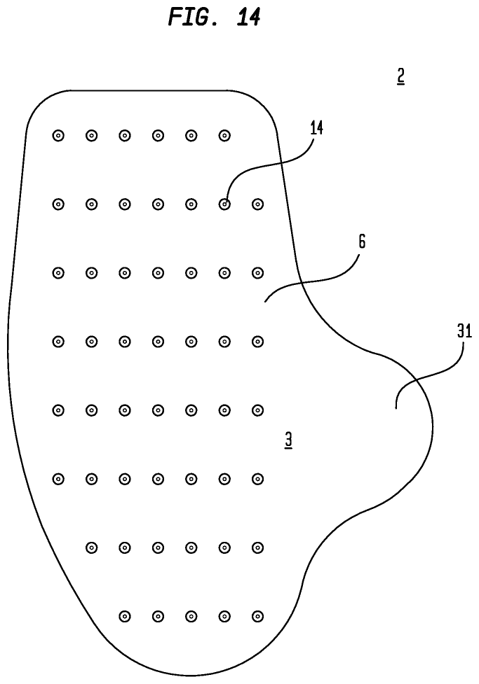

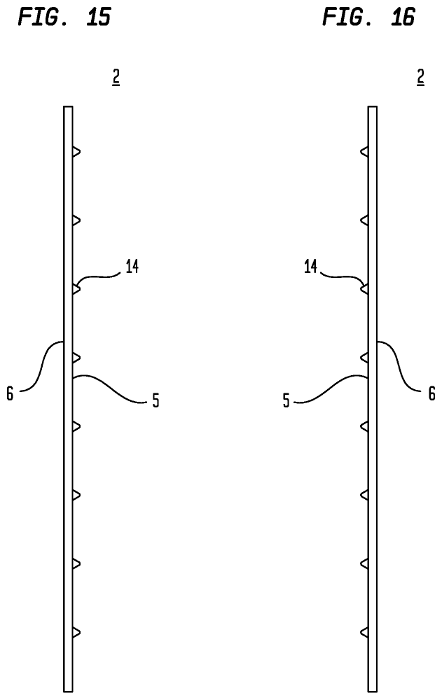

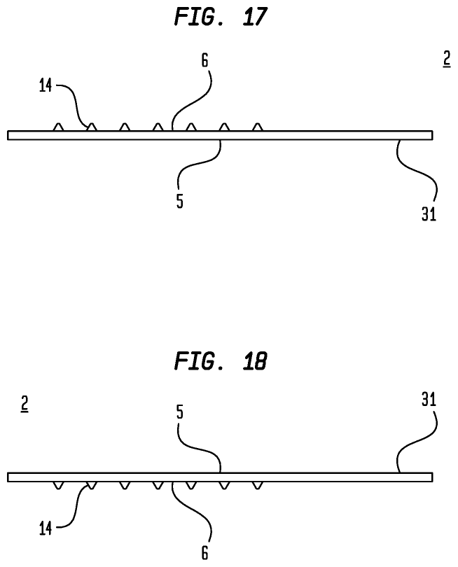

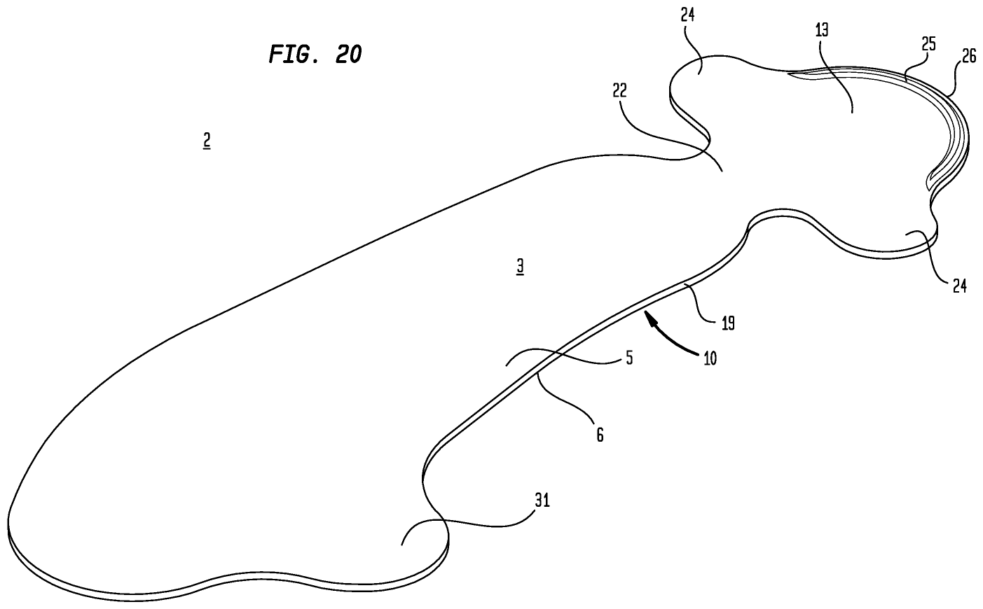

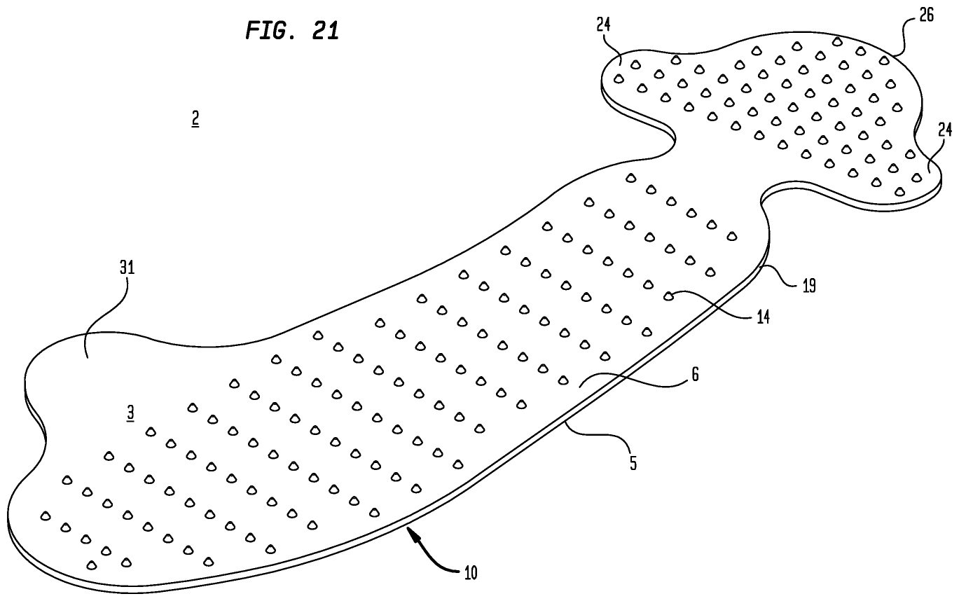

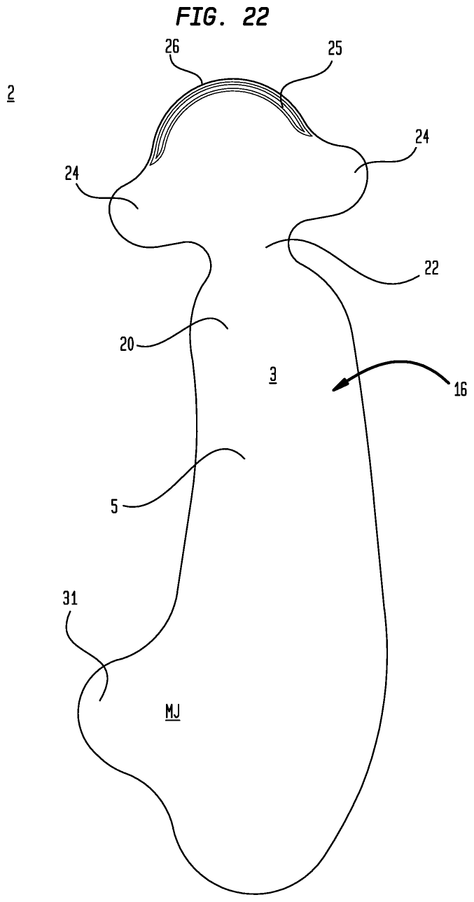

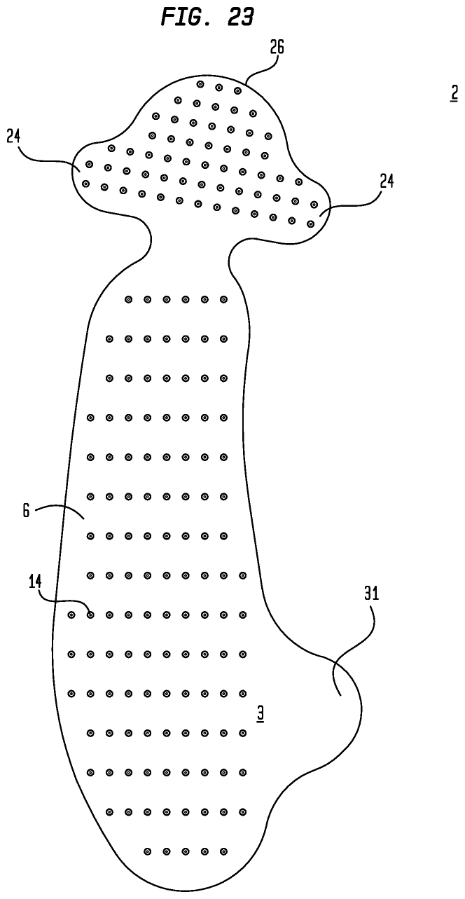

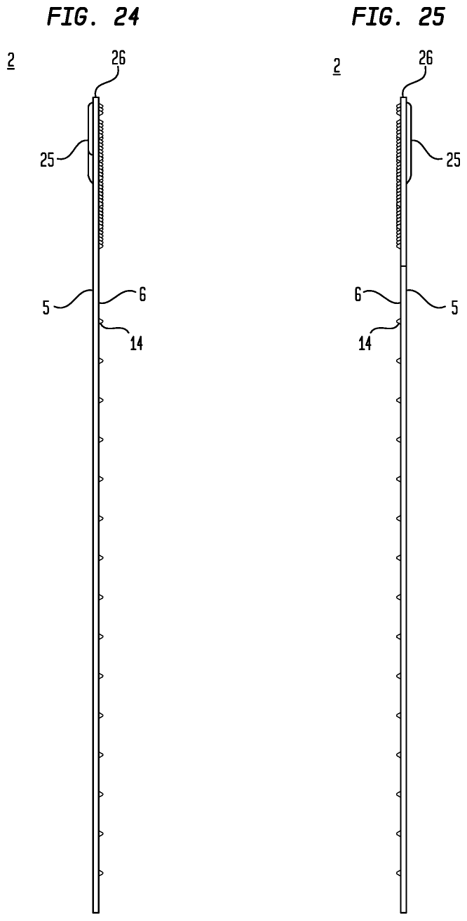

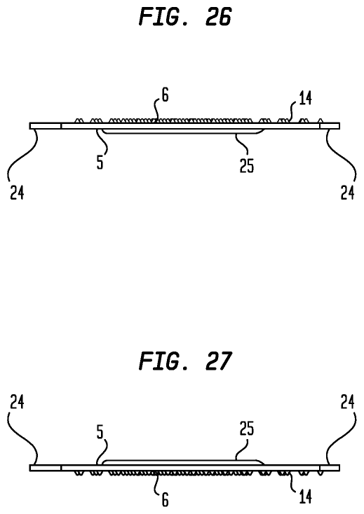

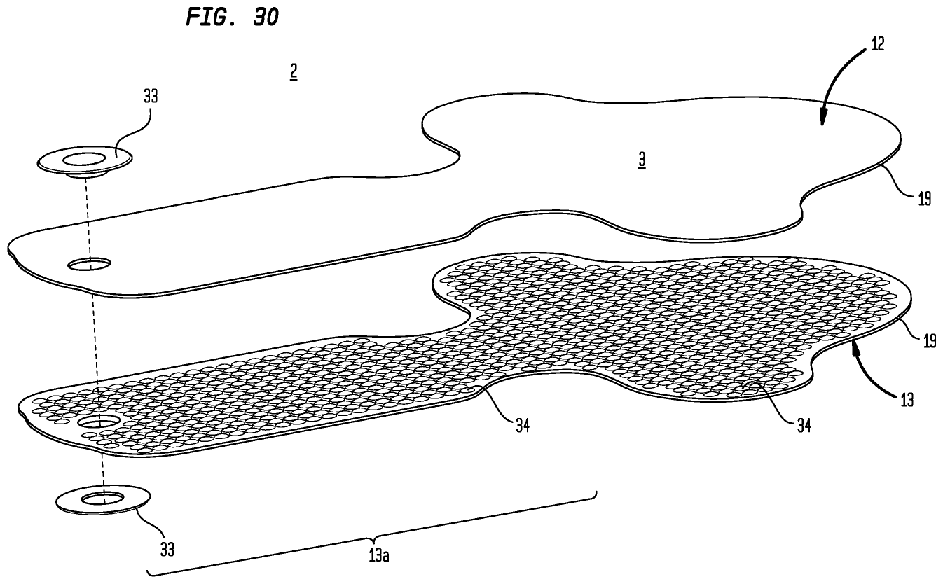

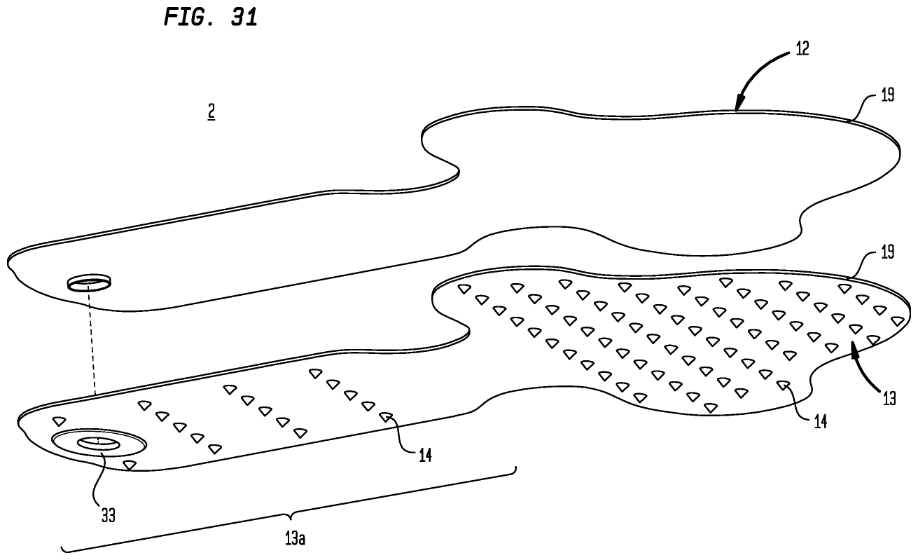

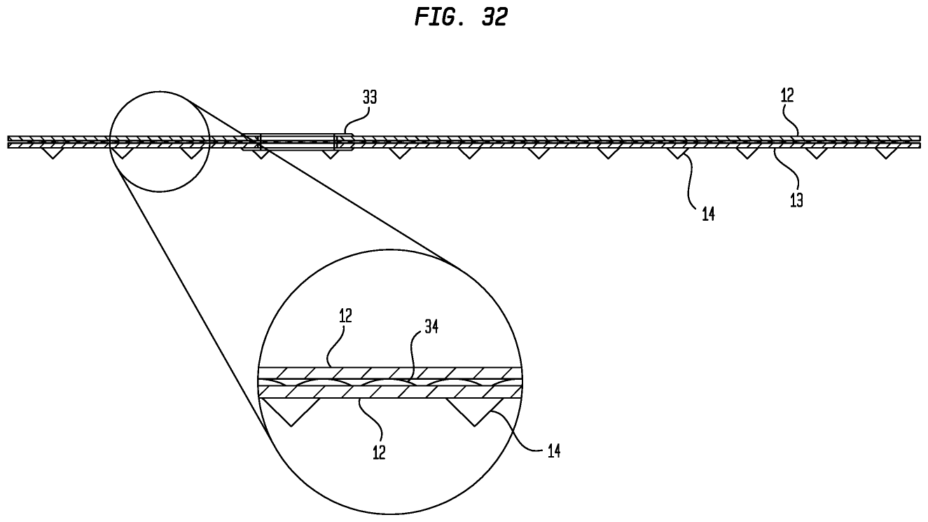

Generally, referring to FIGS. 1 through 32, which depict a blister prevention system (1) including one or more of: a friction barrier (2) adapted to or configured to position between a support surface (SPS) and a skin surface (SS) and methods of making and using the blister prevention system (1) and/or the friction barrier (2).

For the purpose of this invention, the term “friction barrier (2)” means a material disposed between a support surface (SPS) and a skin surface (SS) that reduces at least one of the four mechanical factors contributing to blister development, friction, pressure, shear, and moisture. The term “support surface (SPS)” means any structure adapted to or configured to support or cover a skin surface(S) of an vertebrate animal (A) (depicted in broken line to broadly represent any support structure (SPS), and without limitation to the breadth of the foregoing, includes as examples: a structure adapted to or configured to support or cover a heel portion (H) or plantar (P) portion of a human foot (F), and can further include one or more parts of a shoe (SH) (for example: a heel counter, a vamp, an upper, toe cap, eyestay, eyelets, and tongue); or a structure adapted to or configured to support or cover portion of a palm, one or more fingers or a thumb, and can further include attached one or more parts of glove (for example: a trank, a palm, back of palm, a thumb, one or more quirks, and fourchettes), or a prosthetic structure adapted to or configured to support or cover a residual limb. The term “skin(S)” means the surface of the epidermis and under it the thicker dermis that covers the body of a vertebrate animal (A), including, but not necessarily limited to, a human skin surface. The term “skin surface (SS)” means the external surface of the skin(S) of a vertebrate animal (A), including, but not necessarily limited to, external surface of the human skin. The term “blister” means a fluid filled a space between layers of skin. The term “blister prevention” means reducing at least one of the four mechanical factors contributing to blister development of the skin, friction, pressure, shear, and moisture.

Now, with general reference to, FIGS. 1, 10, 19, and 28 which depict illustrative examples of friction barriers (2) in context of a foot covering or shoe (SH) (depicted in broken line); provided that, the illustrative examples of friction barriers (2) in the context of a shoe (SH) are not intended to preclude embodiments of the inventive friction barrier (2) useful in the context of other types of support surfaces (SS) such as hand covering, inside leg coverings, inside arm coverings, prosthetic sockets, or other support surfaces (SS) that directly or indirectly contact a skin surface (SS).

Now, with general reference to FIGS. 1 through 32, particular embodiments of a friction barrier (2) can comprise a generally flat sheet material (3) generally conformable by placement between the support surface (SPS) and the skin surface (SS). In other particular embodiments, the friction barrier (2) can comprise a pre-formed material (4) adapted to or generally configured to be placed between the support surface (SPS) and the skin surface (SS) (as shown in the illustrative examples of FIGS. 1. 10, 19 and 28. The term “flat sheet material (3)” means a material having a thickness disposed between a generally flat first side (5) opposite a generally flat second side (7). The term “generally” means conformable or pre-formed to an approximation of the shape of the support surface (SPS) or the skin surface (SS) but not in strict conformance to the shape of the support surface (SPS) or the skin surface (SS). The term “facing” means oriented toward but not necessarily directly contacting the support surface (SPS) or the skin surface (SS), for example, the skin surface (SS) may be covered by a thin covering article (7) (for example, a sock over a foot). The term “pre-formed” means formed or shaped in whole or on part before placement between the support surface (SPS) and the skin surface (SS).

Embodiments of the friction barrier (2) can include a friction barrier first side (8) facing the skin surface (SS) and a friction barrier second side (9) facing the support surface (SS). The friction barrier first side (9) facing the skin surface (SS) can have a static coefficient of friction (fr) of less than 0.2. The static coefficient of friction (fr) can be calculated as:

$$fr = Fr / N ,$$

wherein, Fr is the force of friction divided by N the normal or perpendicular force in Newtons (N) pushing two objects together.

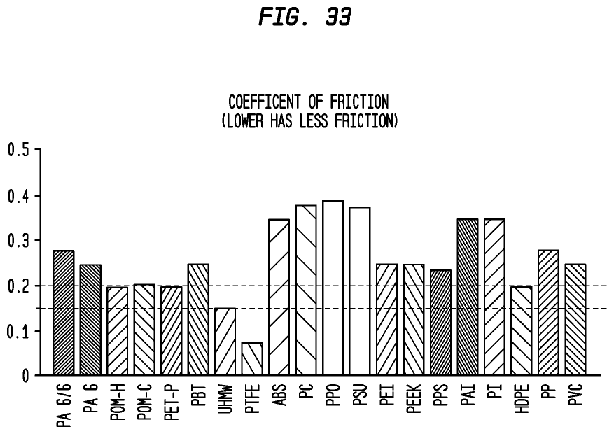

Now, with primary reference to FIG. 33, in particular embodiments, the generally flat sheet material (3) or the pre-formed material (4) can include the friction barrier first side (8) facing the skin surface (SS) having a static coefficient of friction (fr) of less than 0.2. As illustrative examples, the generally flat sheet material (3) or the pre-formed material (4) having a friction barrier first side (8) of (fr) less than 0.2 can comprise, essentially consist of, or consist of, one or more of: polyoxymethylene copolymer, polyoxymethylene homopolymer, polyethylene terephthalate, high-density polyethylene, ultra-high molecular weight polyethylene, polytetrafluoroethylene, fluorinated ethylene propylene, and perfluoroalkoxy alkane. In other particular embodiments, the generally flat sheet material (3) or the pre-formed material (4) can include a friction barrier first side (8) facing the skin surface (SS) having a static coefficient of friction (fr) of less than 0.15. As illustrative examples, the generally flat sheet material (3) or pre-formed material (4) having a friction barrier first side (8) of (fr) less than 0.15 can comprise, essentially consist of, or consist of one or more of: polytetrafluoroethylene, fluorinated ethylene propylene, and perfluoroalkoxy alkane.

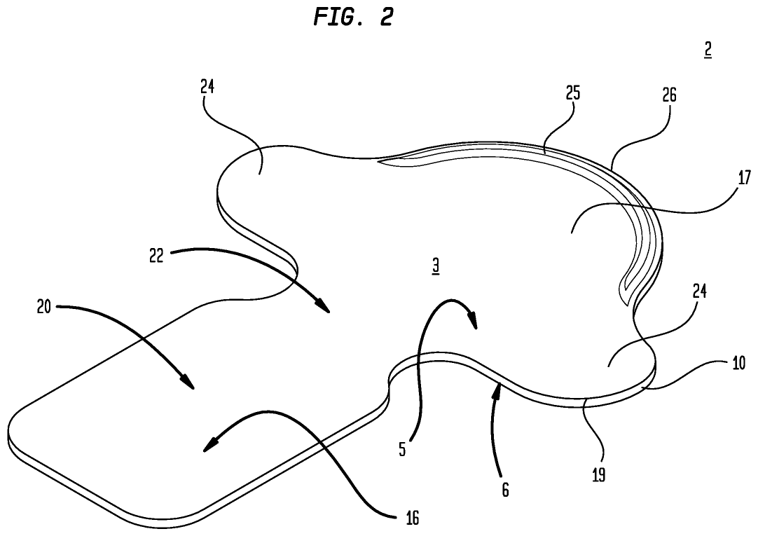

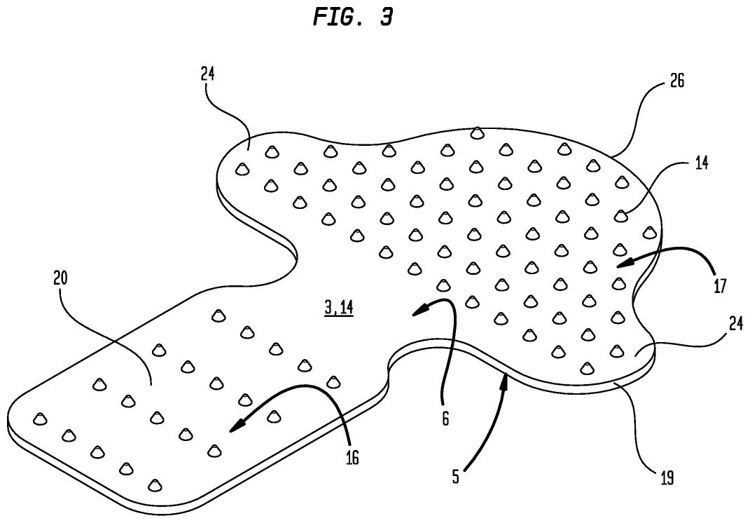

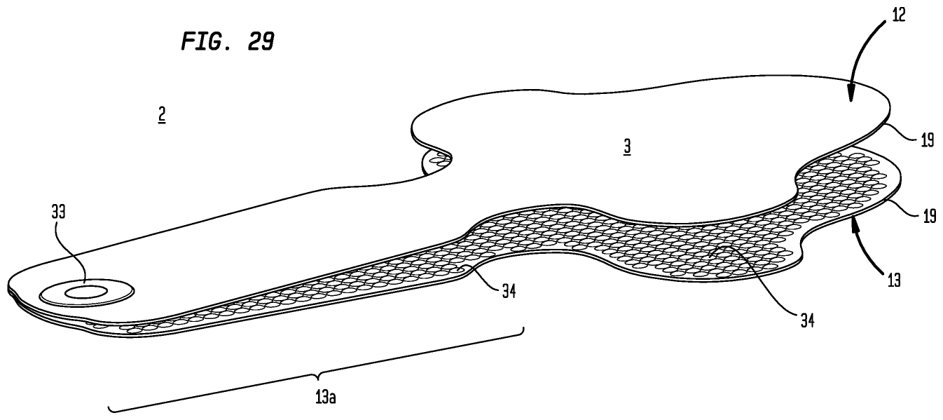

Now, with primary reference to FIGS. 2, 11, 20 and 29 which depict illustrative examples of embodiments of the friction barrier (2) which comprise a generally flat sheet material (3) generally conformable by placement between the support surface (SPS) and the skin surface (SS) as shown in the corresponding illustrative examples of FIGS. 1, 10, 19 and 28. In these particular embodiments, the friction barrier (2) can be provided as generally flat sheet material (3) having a coefficient of friction of less than 0.2 and in certain instances less than 0.15 with a thickness of between about 15 mils (0.015 inch, about 0.38 millimeter) and 78 mils (0.078 inch, about 2 millimeters). The generally flat sheet material (3) can comprise, essentially consist of, or consist of only one layer (10) of one generally flat sheet material (3). While in other embodiments, the generally flat sheet material (3) can comprise a plurality of layers (11) (as shown in the illustrative example of FIG. 29) having the friction barrier first side (8) facing the skin surface (SS) having a coefficient of friction of less than 0.2, or in certain embodiments less than 0.15. In particular embodiments, the first layer (12) and the second layer (13) can be integrated into one piece, while in other embodiments, the first layer (12) and the second layer (13) can be separate discrete layers capable or adapted to move in relation to one another.

As shown in the illustrative examples of 3, 12 and 23, the friction barrier second side (9) can, but need not necessarily, include raised surface elements (14) adapted to or configured to resist movement of the friction barrier second side (6) relative to the support surface (SPS). The generally flat sheet material (3) can be processed to produce copies of a plurality of cut-out shapes (for example by: die cutting or laser cutting) that can be placed between the support surface (SPS) and the skin surface (SS). The cut-out flat sheet material (14) can have the friction barrier second side (6) disposed facing the support surface (SPS). The skin surface (SS) facing the friction barrier first side (5) can be pressed against of the generally flat sheet material (3) (cut-out flat sheet material (14)) to generally conform the friction barrier first side (5) of the generally flat sheet material (4) to the skin surface (SS) to produce embodiments of the friction barrier (2) as shown in the illustrative examples of FIGS. 1, 10, 19 and 28.

Again, with general reference to FIGS. 1, 10, 19, and 28 particular embodiments of the friction barrier (2) can comprise a pre-formed material (4) placed between the support surface (SPS) and the skin surface (SS), wherein the friction barrier (2) has friction barrier first side (8) facing he skin surface (SS) having a static coefficient of friction (fr) of less than 0.2, and in certain embodiments less than 0.15, as above described. While certain materials such as: polytetrafluoroethylene, fluorinated ethylene propylene, and perfluoroalkoxy alkane will not flow above their respective melting points, these materials cannot be injection molded but can pre-formed into friction barriers (2) described herein by compression molding of the powdered material into preforms, and then sintering the preforms in a process analogous to sintered metal processing. This process results in pre-formed materials (4) having shapes that can be disposed as friction barriers (2) between a support surface (SPS) and a skin surface (SS).

Now, with primary reference to FIGS. 1 through 9, a particular embodiment of the friction barrier (2) can be generally conformed by pressing engagement or by being pre-formed to the shape of the heel (H) of the human foot (F). As shown in the illustrative example of FIG. 1, the friction barrier second side (9) can be disposed in the heel portion (HP) of a shoe (SH) with the friction barrier first side (8) facing the skin surface (SS) of the heel (H) of the foot (F) when received in the shoe (SH). In particular embodiments, the friction barrier (2) can include a plantar heel portion (16) joined to a back heel portion (17). The plantar heel portion (16) can be disposed against the inner sole (18) of the shoe (SH). The illustrative example of FIGS. 1 through 9, depicts the plantar heel portion (16) extending to a friction barrier perimeter (19) to delimit a heel portion area (20) of a substantially rectangular configuration; however, this is not intended to delimit the plantar heel portion (16) to any particular contour of the friction barrier perimeter (19) and the friction barrier perimeter (19) can delimit any heel portion area (16) that allows insertion of the plantar heel portion (16) of the friction barrier (2) into the shoe (SH) to be disposed against the inner sole (18) of the shoe (SH), in certain embodiments, the plantar heel portion (16) may extend to cover a portion of a shoe upper (21) of the shoe (SH).

Again, with primary reference to FIGS. 1 through 9, the friction barrier perimeter (19) can narrow approaching the back heel portion (17) to provide a bendable area (22) in the generally flat sheet material (3) to allow the back heel portion (17) to be disposed in generally upright relation to the plantar heel portion (16) and against the inside heel counter (23) of the shoe (SH). In particular embodiments, back heel portion (17), but need not necessarily include, an ankle cover (24) outwardly extending from the back heel portion (17) to face a skin surface (SS) of an inner ankle (IA) or an outer ankle (OA) or both the inner and outer ankle (IA, OA) of the foot (F). In particular embodiments, the friction barrier (2) can, but need not necessarily, include a raised bead (25) proximate the terminal upper edge (26) of the back heel portion (17). In the illustrative example of FIG. 2, the back heel portion (17) defines a curved terminal upper edge (26) with the raised bead (25) disposed along the margin of the curved terminal upper edge (26). The raised bead (26) can be raised outward of the back heel portion (17) by about 40 mil to 120 mil (1 millimeter to 3 millimeters) to resist movement of the (SS) beyond the terminal upper edge (26) of the friction barrier (2). The force of subsequent pressed engagement of the skin surface (SS) of the heel (H) of the foot (F) against the friction barrier first side (5) of the friction barrier (2) can generally conform the flat sheet material (3) to the heel (H) of the foot (F). The resultant friction barrier (2) generally conformed to the heel (H) of the foot (F) can remain in the shoe (SH) or be repeatedly removed and refitted to the shoe (SH).

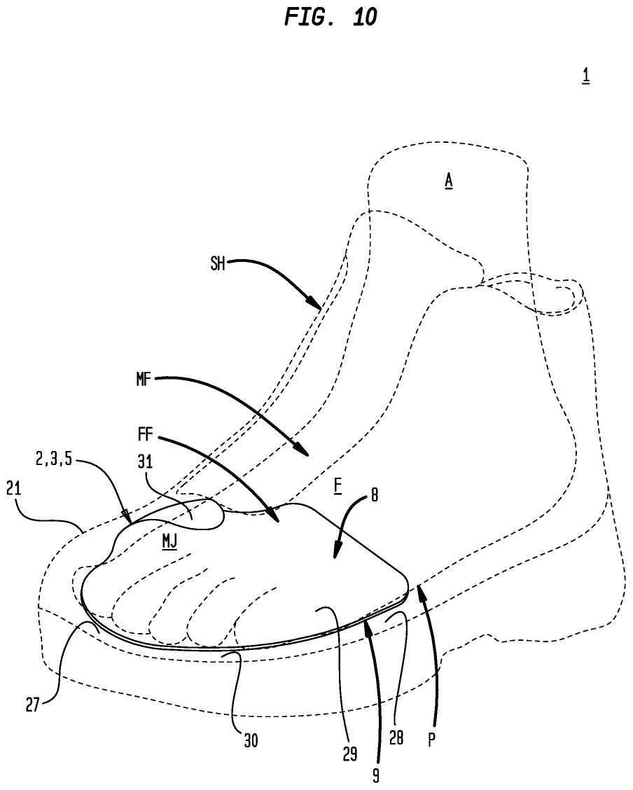

Now, with primary reference to FIGS. 10 through 18, particular embodiments of the friction barrier (2) can comprise a generally flat sheet material (3) generally conformable by placement between a support surface (SPS) and the skin surface (SS) of a plantar (P) forefoot (FF) of a foot (F) or a pre-formed material (5) for placement between the support surface (SS) and the skin surface (SS) of a plantar (P) forefoot (FF) of the foot (F). As shown in FIG. 10, the friction barrier second side (9) of the friction barrier (2) can be disposed in the forefoot portion (FFP) of a shoe (SH) with the friction barrier first side (8) facing the skin surface (SS) of the forefoot (FF) of the foot (F) when the foot (F) is received in the shoe (SH). The friction barrier second side (9) can be disposed against the forefoot inner sole (30) of the shoe (SH). The illustrative example of FIGS. 10 through 18, depicts the friction barrier (2) having a friction barrier perimeter (19) generally contoured to the shape of the inner sole (18) of the shoe (SH) extending from proximate a toe end inner sole (27) to about the midfoot inner sole (28); however, this is not intended to delimit the friction barrier (2) to any particular contour of the inner sole (18) and the friction barrier perimeter (19) can delimit any forefoot area (29) that allows insertion of the friction barrier (2) into the forefoot (FF) of the shoe (SH) to be disposed against the forefoot inner sole (30) of the shoe (SH), in certain embodiments, the friction barrier (2) may extend to cover a portion of the shoe upper (21) of the shoe (SH). In particular embodiments, a big toe cover (31) can outwardly extend from the medial side of the friction barrier (2) to face a skin surface (SS) covering the metatarsophalangeal joint (MJ) of the foot (F). The force of subsequent pressed engagement of the skin surface (SS) of the forefoot (FF) of the foot (F) against the friction barrier first side (8) can generally conform the flat sheet material (3) to the forefoot (FF) of the foot (F). The resultant friction barrier (2) generally conformed to the forefoot (FF) of the foot (F) can remain in the shoe (SH) or be repeatedly removed and refitted to the shoe (SH).

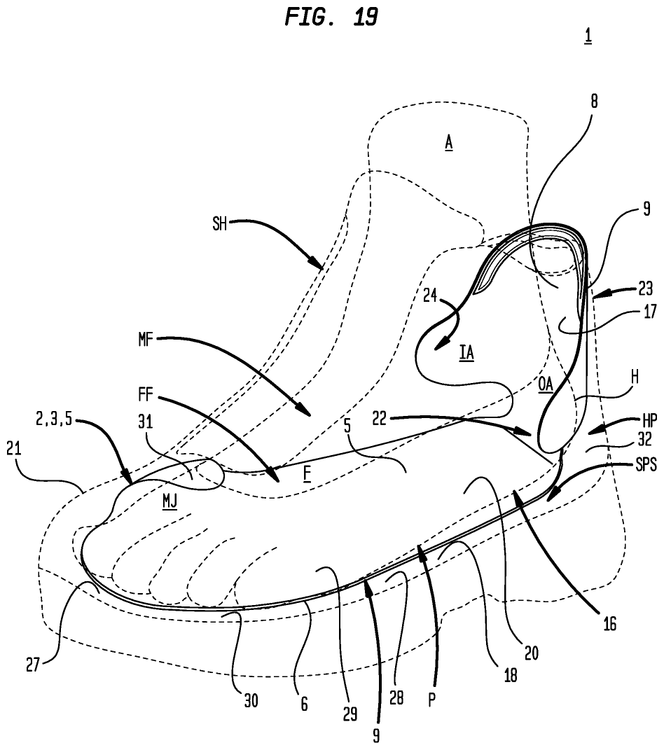

Now, with primary reference to FIGS. 19 through 27, particular embodiments of the friction barrier (2) can comprise a generally flat sheet material (3) generally conformable by placement between a support surface (SS) and a combined skin surface (SS) of a plantar forefoot (FF), plantar midfoot (MF), and heel (H) of the foot (F). As shown in FIG. 19, the friction barrier second side (9) of the friction barrier (2) can be disposed in a shoe (SH) with the friction barrier first side (8) facing the skin surface (SS) of the plantar forefoot (FF), plantar midfoot (PM), and heel (H) of the foot (F) when the foot (F) is received in the shoe (SH). The friction barrier second side (9) can be disposed against the combination of forefoot inner sole (30), the midfoot inner sole (28) and the heel (H) of the shoe (SH). The illustrative example of FIGS. 10 through 18, depicts the friction barrier (2) having a friction barrier perimeter (19) generally contoured to the shape of the inner sole (18) of the shoe (SH) extending from proximate the toe end inner sole (27) to the heel end inner sole (32); however, this is not intended to delimit the friction barrier (2) to any particular contour of the inner sole (18) and the friction barrier perimeter (19) can delimit any inner sole area that allows insertion of the of the friction barrier (2) into the shoe (SH) to be disposed against the inner sole (18) of the shoe (SH), in certain embodiments, the friction barrier (2) may extend to cover a portion of the shoe upper (21) of the shoe (SH). Particular embodiments can further include an ankle cover (24) and/or a big toe cover (31), as above described. The force of subsequent pressed engagement of the combined skin surface (SS) of the plantar forefoot (FF), the plantar midfoot (MF) and the heel (H) of the foot (F) against the generally flat first side (5) of the generally flat sheet material (3) can generally conform the flat sheet material (3) to the combination of the plantar forefoot (FF), plantar midfoot (MF), and heel (H) of the foot (F). The resultant friction barrier (2) generally conformed to the plantar forefoot (FF), plantar midfoot (MF), and heel (H) of the foot (F) can remain in the shoe (SH) or be repeatedly removed and refitted to the shoe (SH).

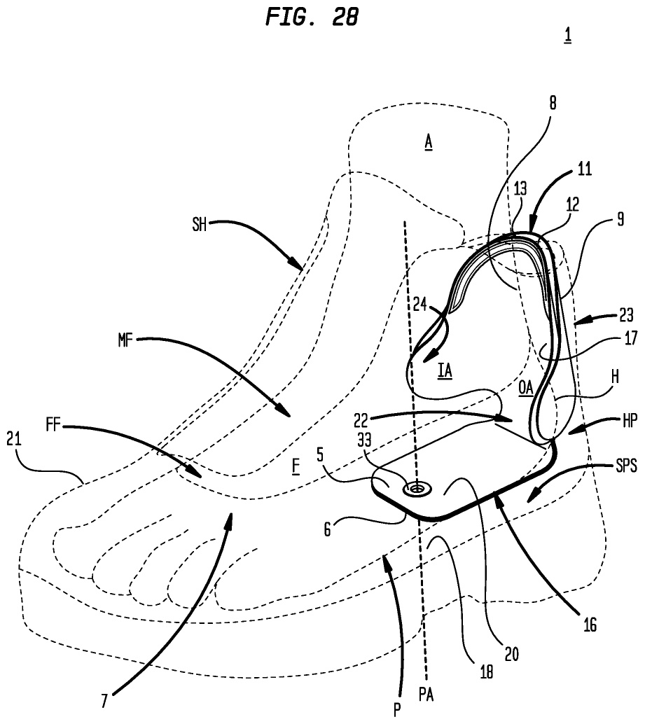

Now, with primary reference to FIGS. 28 through 32, in particular embodiments of the friction barrier (2), the generally flat sheet material (3) generally conformable by placement between the support surface (SPS) and said skin surface (SS) or the pre-formed material (4) placed between the support surface (SPS) and the skin surface (SS) can comprise a plurality of layers (11), wherein a first layer (12) includes the friction barrier first side (8) facing the skin surface (SS), and wherein the second layer (13) includes the friction barrier second side (9) facing the support surface (SS), wherein said first layer (12) facing the skin surface (SS) having a static coefficient of friction of less than 0.2, or even less than 0.15. While the illustrative example of FIGS. 28 through 32, depicts a friction barrier (2) adapted to or configured to the shape of the heel (H) of the human foot (F); this is not intended to preclude embodiments of the friction barrier (2) having a plurality of layers (11) which can be adapted to or configured to be placed between a wide variety of support surfaces (SPS) and corresponding skin surfaces (SS), as above describe. In particular embodiments, the first layer (12) and the second layer (13) can be integrated as one piece with the first layer (12) disposed in fixed immovable spatial relation to the second layer (13). In the illustrative examples of 28 through 32, the first layer (12) remains discrete from or generally detached from the second layer (13) allowing the first layer (12) to slidably move in relation to the second layer (13).

Now, with primary reference to the examples of FIGS. 29 through 32, the first layer (12) and the second layer (13) can be separate layers attached by a pivot element (33) allowing the first layer (12) to rotate relative to the second layer (13) about a pivot axis (PA). In particular embodiments, raised antifriction surface elements (34) disposed on the support surface (SPS) facing side of the first layer (12) or disposed on the skin surface (SS) facing side of the second layer (13) can be adapted or configured to reduce friction forces between the first layer (12) and the second layer (13). In particular embodiments, the first layer (12) facing the skin surface (SS) can have a static coefficient of friction of less than 0.2, and as illustrative examples, can comprise, essentially consist of, or consist of one or more of: high-density polyethylene and ultra-high molecular weight polyethylene. In other particular embodiments, the first layer (12) facing the skin surface (SS) can have a static coefficient of friction of less than 0.15, and as illustrative examples, can comprise, essentially consist of or consist of one or more of: polytetrafluoroethylene, fluorinated ethylene propylene, and perfluoroalkoxy alkane. The second layer (13) facing the support surface (SPS) can have coefficient of friction (fr) greater than 0.15 and in particular embodiments greater than 0.2. The second layer (13) can comprise one or more of: polybutylene terephthalate, acrylonitrile butadiene styrene, polycarbonate, poly(p-phenylene oxide), polysulfone, polyetherimide, polyether ether ketone, polyphenylene sulfide, polyamide-imide, polypropylene, polyvinyl chloride, nylon 66, polyamide nylon, polyoxymethylene copolymer, polyoxymethylene homopolymer, polyethylene terephthalate, high-density polyethylene, ultra-high molecular weight polyethylene, polytetrafluoroethylene, fluorinated ethylene propylene, and perfluoroalkoxy alkane.

Now, with primary reference to FIGS. 28 through 31, in particular embodiments, the second layer (13) can comprise a partial second layer (13a). A partial second layer (13a) can comprise any portion of the second layer (13) from the second layer end proximate the pivot element (33) to the bendable area (22) in the generally flat sheet material (3) (as shown in the example of FIG. 28 and by the broken line in the examples of FIGS. 29 through 31).

As can be easily understood from the foregoing, the basic concepts of the present invention may be embodied in a variety of ways. The invention involves numerous and varied embodiments of a friction barrier system (1) and a friction barrier (2) and methods for making and using such friction barrier system (1) and friction barrier (2) including the best mode.

As such, the particular embodiments or elements of the invention disclosed by the description or shown in the figures or tables accompanying this application are not intended to be limiting, but rather exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of a “connector” should be understood to encompass disclosure of the act of “connecting”—whether explicitly discussed or not—and, conversely, were there is a disclosure of the act of “connecting”, such a disclosure should be understood to encompass disclosure of a “connector” and even a “means for connecting”. Such alternative terms for each element or step are to be understood to be explicitly included in the description.

In addition, as to each term used it should be understood that unless its utilization in this application is inconsistent with such interpretation, common dictionary definitions should be understood to be included in the description for each term as contained in the Random House Webster's Unabridged Dictionary, second edition, each definition hereby incorporated by reference.

All numeric values herein are assumed to be modified by the term “about”, whether or not explicitly indicated. For the purposes of the present invention, ranges may be expressed as from “about” one particular value to “about” another particular value. When such a range is expressed, another embodiment includes from the one particular value to the other particular value. The recitation of numerical ranges by endpoints includes all the numeric values subsumed within that range. A numerical range of one to five includes for example the numeric values 1, 1.5, 2, 2.75, 3, 3.80, 4, 5, and so forth. It will be further understood that the endpoints of each of the ranges are significant both in relation to the other endpoint, and independently of the other endpoint. When a value is expressed as an approximation by use of the antecedent “about,” it will be understood that the particular value forms another embodiment. The term “about” generally refers to a range of numeric values that one of skill in the art would consider equivalent to the recited numeric value or having the same function or result. Similarly, the antecedent “substantially” means largely, but not wholly, the same form, manner or degree and the particular element will have a range of configurations as a person of ordinary skill in the art would consider as having the same function or result. When a particular element is expressed as an approximation by use of the antecedent “substantially,” it will be understood that the particular element forms another embodiment.

Moreover, for the purposes of the present invention, the term “a” or “an” entity refers to one or more of that entity unless otherwise limited. As such, the terms “a” or “an”, “one or more” and “at least one” can be used interchangeably herein.

Further, for the purposes of the present invention, the term “coupled” or derivatives thereof can mean indirectly coupled, coupled, directly coupled, connected, directly connected, or integrated with, depending upon the embodiment.

Additionally, for the purposes of the present invention, the term “integrated” when referring to two or more components means that the components (i) can be united to provide a one-piece construct, a monolithic construct, or a unified whole, or (ii) can be formed as a one-piece construct, a monolithic construct, or a unified whole. Said another way, the components can be integrally formed, meaning connected together so as to make up a single complete piece or unit, or so as to work together as a single complete piece or unit, and so as to be incapable of being easily dismantled without destroying the integrity of the piece or unit.

Thus, the applicant(s) should be understood to claim at least: i) each of the friction barrier systems or friction barriers herein disclosed and described, ii) the related methods disclosed and described, iii) similar, equivalent, and even implicit variations of each of these devices and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The background section of this patent application, if any, provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification, if any, are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent application or continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon. The elements following an open transitional phrase such as “comprising” may in the alternative be claimed with a closed transitional phrase such as “consisting essentially of” or “consisting of” whether or not explicitly indicated the description portion of the specification.

Additionally, the claims set forth in this specification, if any, are further intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

1. A blister prevention apparatus, comprising:
a friction barrier adapted to position between a support surface and a skin surface, wherein said friction barrier comprises a generally flat sheet material generally conformable by placement between said support surface and said skin surface, or a pre-formed material placed between said support surface and said skin surface, wherein said friction barrier has first side adapted to face said skin surface having a static coefficient of friction (fr) of less than 0.2.

2. The apparatus of claim 1, wherein (fr) is calculated as:
$$fr = Fr / N ,$$
wherein Fr is the force of friction divided by N the normal or perpendicular force in Newtons pushing two objects together.

3. The apparatus of claim 1, wherein said generally flat sheet material comprises or essentially consists of one material.

4. The apparatus of claim 1, wherein said friction barrier has a second side adapted to face said support surface, said second side including raised surface elements.

5. The apparatus of claim 4, wherein said raised surface elements configured to resist movement of said second side relative to said support surface.

6. The apparatus of claim 1, wherein said friction barrier having said first side adapted to face said skin surface having a static coefficient of friction (fr) of less than 0.2 comprises one or more of: high-density polyethylene and ultra-high molecular weight polyethylene.

7. The apparatus of claim 1, wherein said first side adapted to face said skin surface having a static coefficient of friction (fr) of less than 0.15.

8. The apparatus of claim 7, wherein said first side adapted to face said skin surface having a static coefficient of friction (fr) of less than 0.15 comprises one or more of:
polytetrafluoroethylene, fluorinated ethylene propylene, and perfluoroalkoxy.

9. The apparatus of claim 1, wherein said generally flat sheet material generally conformable by placement between said support surface and said skin surface. or said pre-formed material placed between said support surface and said skin surface, comprises a plurality of layers, wherein a first layer including said first side adapted to face said skin, and wherein said second layer including said second side adapted to face said support surface, wherein said first layer adapted to face said skin surface having said static coefficient of friction of less than 0.2.

10. The apparatus of claim 9, wherein said first layer and said second layer integrated as one piece.

11. The apparatus of claim 9, wherein said first layer and said second layer each comprise a separate discrete layer.

12. The apparatus of claim 11, wherein said first layer slidably engages said second layer.

13. The apparatus of claim 12, wherein said second layer pivotally attached to said first layer.

14-18. (canceled)

19. The apparatus of claim 1, wherein said friction barrier configured to generally conform to said skin surface of a human foot.

20. The apparatus of claim 19, wherein said friction barrier configured to generally conform to said skin surface of a heel of a human foot.

21. The apparatus of claim 20, wherein said friction barrier configured to generally conform to said skin surface of said heel of said human foot, includes a plantar heel portion and a back heel portion.

22. The apparatus of claim 20, further comprising an ankle cover portion outwardly extending from said back heel portion to cover a skin surface of an inner ankle of said human foot or a skin surface of an outer ankle, and a combination thereof.

23. The apparatus of claim 21, further comprising a raised bead proximate the terminal edge of said back heel portion.

24. The apparatus of claim 19, wherein said friction barrier configured to generally conform to said skin surface of a plantar forefoot of said human foot.

25. The apparatus of claim 24, wherein said friction barrier generally configured to generally conform to a combination of said plantar forefoot and a plantar midfoot of said human foot.

26. The apparatus of claim 25, wherein said friction barrier generally configured to generally conform to a combination of said plantar forefoot, said plantar midfoot, and a heel: of said human foot.

27-78. (canceled)
